(19) United States
(12) Patent Application Publication
(10) Pub. No.: US 2024/0257968 A1
(43) Pub. Date: Aug. 1, 2024

(54) AUTOMATED NEUROLOGICAL ANALYSIS SYSTEMS AND METHODS

(71) Applicant: DANE HEALTH, INC., Huntington Beach, CA (US)

(72) Inventors: Nirav Patel, Huntington Beach, CA (US); David Schneider, Scottsdale, AZ (US); Sanjay Joshi, Kirkland, WA (US)

(21) Appl. No.: 18/423,930

(22) Filed: Jan. 26, 2024

Related U.S. Application Data
(60) Provisional application No. 63/481,658, filed on Jan. 26, 2023.

(51) Int. Cl. G16H 50/20; G16H 10/20; G16H 10/60; G16H 15/00; G16H 50/30

(52) U.S. Cl. CPC G16H 50/20; G16H 10/20; G16H 10/60; G16H 15/00; G16H 50/30

(57) ABSTRACT

Systems and methods related to the automated neurological analysis of patients are disclosed that can enable individuals to more easily and conveniently obtain assessments of potential neurological impairments that they may be suffered from, as well as enabling the collection and sharing of medical data that can help with such assessments. The overall treatment of the patient can be optimized and enhanced, as well as providing education and directions to the patient.

### CROSS-REFERENCE TO RELATED APPLICATION

This application claims priority to U.S. Provisional Patent App. No. 63/481,658 filed on Jan. 26, 2023, the contents of which are incorporated herein in its entirety.

### TECHNICAL FIELD

This application generally relates to systems and methods for the automated neurological analysis of patients. In particular, this application relates to the assessment of patients to identify potential neurological conditions and disorders through the use of an application executing on a computing device, including the collection, analysis, storage, and/or transmission of relevant medical data.

### BACKGROUND

Individuals may experience collisions, falls, and other events that can result in a variety of injuries and/or impairments. These events may occur during vehicle collisions, home and work accidents, or sports-related activities, for example, and can result in an individual suffering from a neurological impairment, such as a concussion. Injured individuals may not have the means or ability to physically visit or consult with a doctor or other medical provider for diagnosis and treatment. For example, the individuals may not have an existing relationship with a suitable doctor, or may not know how to find a suitable doctor in their area.

Therefore, there is a need for systems and methods to enable individuals to more easily and conveniently obtain assessments and identifications of potential neurological impairments that they may be suffering from, as well as enabling the collection and sharing of medical data that can help with such assessments and identifications.

### SUMMARY

The invention is intended to solve the above-noted problems and other problems by providing systems and methods for the assessment of patients to identify potential neurological conditions and disorders through the use of an application executing on a computing device, including the collection, analysis, storage, and/or transmission of relevant medical data. The invention includes performing a neurological assessment using the computing device, including through the collection and analysis of medical data and/or sensor data. The assessment may determine a result based on whether a criteria is satisfied, such as an urgent result, positive non-urgent result, or negative non-urgent result related to the patient's neurological condition. For positive non-urgent results, an identification of a potential neurological impairment of the patient can be determined. Regardless of the result, various messages can be displayed to the patient to educate and/or direct the patient. In addition, the patient's medical data and/or sensor data can be transmitted and stored for use and analysis by medical providers, insurance companies, etc.

These and other embodiments, and various permutations and aspects, will become apparent and be more fully understood from the following detailed description and accompanying drawings, which set forth illustrative embodiments that are indicative of the various ways in which the principles of the invention may be employed.

### BRIEF DESCRIPTION OF THE DRAWINGS

FIG. 1 is a block diagram of an exemplary system for neurological analysis of patients, including a computing device in communication with a remote server, in accordance with some embodiments.

FIG. 2 is a flowchart illustrating operations for the neurological analysis of patients, using the system of FIG. 1, in accordance with some embodiments.

### DETAILED DESCRIPTION

The description that follows describes, illustrates and exemplifies one or more particular embodiments of the invention in accordance with its principles. This description is not provided to limit the invention to the embodiments described herein, but rather to explain and teach the principles of the invention in such a way to enable one of ordinary skill in the art to understand these principles and, with that understanding, be able to apply them to practice not only the embodiments described herein, but also other embodiments that may come to mind in accordance with these principles. The scope of the invention is intended to cover all such embodiments that may fall within the scope of the appended claims, either literally or under the doctrine of equivalents.

It should be noted that in the description and drawings, like or substantially similar elements may be labeled with the same reference numerals. However, sometimes these elements may be labeled with differing numbers, such as, for example, in cases where such labeling facilitates a more clear description. Additionally, the drawings set forth herein are not necessarily drawn to scale, and in some instances proportions may have been exaggerated to more clearly depict certain features. Such labeling and drawing practices do not necessarily implicate an underlying substantive purpose. As stated above, the specification is intended to be taken as a whole and interpreted in accordance with the principles of the invention as taught herein and understood to one of ordinary skill in the art.

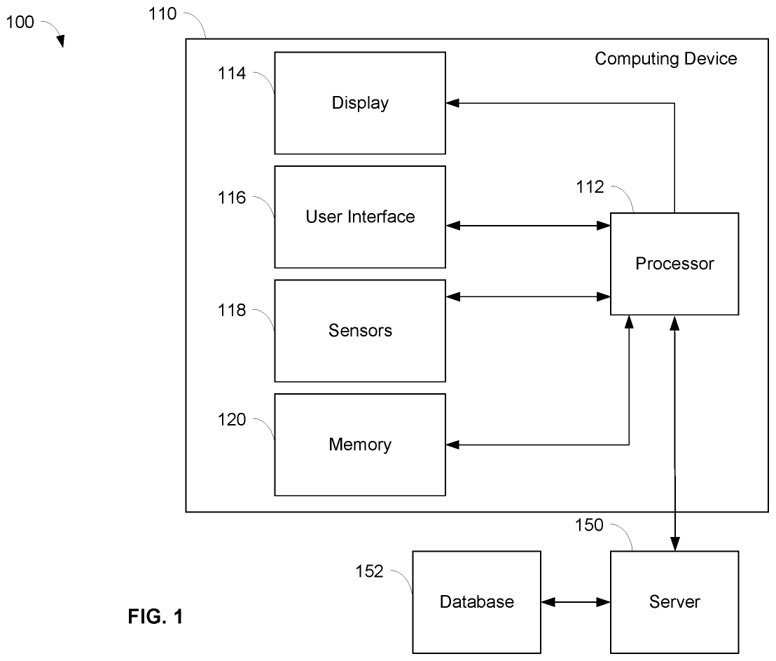

FIG. 1 illustrates a neurological analysis system 100 in accordance with one or more principles of the invention. The system 100 may include modules and components that are connected through a network such as the Internet, which can facilitate communications through secure channels. The system 100 may utilize data received, sensed, and/or collected from a patient to enable the assessment and identification of the patient's neurological condition. In embodiments, the system 100 may enable a patient to communicate with a medical provider in conjunction with the execution of a software application related to the neurological analysis of the patient.

Through use of the system 100, the overall treatment of the patient can be optimized and enhanced, as well as to provide education and directions to the patient. Subsequent visits and consultations with medical providers can also be made more efficient by, for example, enabling the medical providers to have access to a greater amount of patient data, e.g., patient data collected by the system 100 at various times prior to the physical visit. This can result in helping the medical provider to make more appropriate care decisions for the patient. As a result, the patient can be assessed and treated more expeditiously to prevent chronic conditions from developing.

In embodiments, some or all of the software application related to the neurological analysis of the patient may be stored and/or be executable on the computing device 110. In other embodiments, some or all of the software application may be stored and/or be executable on a remote server, e.g., server 150. In further embodiments, some or all of the software application may execute on standard web browsers, such as Chrome, Safari, Firefox, etc.

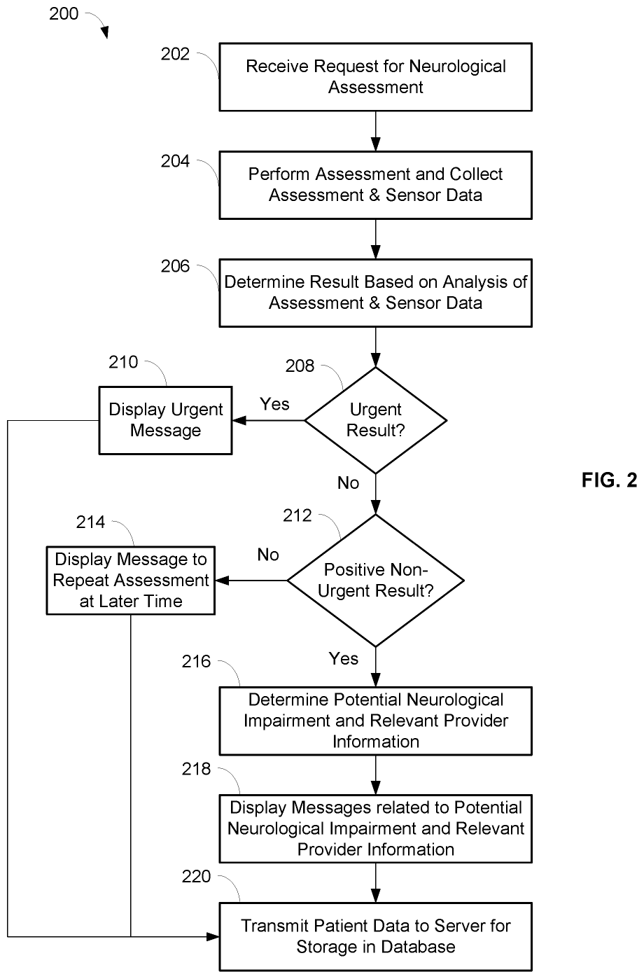

An exemplary embodiment of a process 200 for the system 100 is shown in FIG. 2. One or more processors (e.g., processor 112 and/or other processing components (e.g., analog to digital converters, encryption chips, etc.) within or external to the computing device 110 may perform any, some, or all of the steps of the process 200. One or more other types of components (e.g., display 114, user interface 116, sensors 118, memory 120, transmitters, receivers, buffers, drivers, discrete components, etc.) may also be utilized in conjunction with the processors and/or other processing components to perform any, some, or all of the steps of the process 200. The computing device 110 may be, for example, a personal computer (PC), a laptop, a tablet, a mobile device, a thin client, or other computing platform. The computing device 110 may operate using a suitable operating system, such as Windows, Mac OS, iOS, and Android.

The various components of the computing device 110 may be communicatively coupled by a system bus, network, or other connection mechanism. The processor 112 may include a general purpose processor (e.g., a microprocessor) and/or a special purpose processor (e.g., a digital signal processor (DSP)). The processor 112 may be any suitable processing device or set of processing devices such as, but not limited to, a microprocessor, a microcontroller-based platform, an integrated circuit, one or more field programmable gate arrays (FPGAs), and/or one or more application-specific integrated circuits (ASICs).

The user interface 116 may facilitate interaction with a user of the device. As such, the user interface 116 may include input components such as a keyboard, a keypad, a mouse, a touch-sensitive panel, a sound speaker, or a haptic feedback system. The user interface 116 may also comprise devices that communicate with inputs or outputs, such as a short-range transceiver (RFID, Bluetooth, etc.), a telephonic interface, a cellular communication port, a router, or other types of network communication equipment. The user interface 116 may be internal to the computing device 110, or may be external and connected wirelessly or via connection cable, such as through a universal serial bus port. The display 114 may include a screen, which for example, may be combined with a touch-sensitive panel. The sensors 118 may be internal and/or external to the system 100 and may include, for example, microphones, cameras, motion sensors (e.g., accelerometers), infrared sensors, lights and light sensors, gyroscopes, and/or other suitable sensors.

In embodiments, the sensors 118 may also include biomedical devices such as pressure sensors, eye trackers, electroencephalograph (EEG) devices, electromyograph (EMG) devices, and/or augmented reality/extended reality (AR/XR) devices. The sensors 118 may communicate with the processor 112 using a suitable application programming interface (API).

The memory 120 may be volatile memory (e.g., RAM including non-volatile RAM, magnetic RAM, ferroelectric RAM, etc.), non-volatile memory (e.g., disk memory, FLASH memory, EPROMs, EEPROMs, memristor-based non-volatile solid-state memory, etc.), unalterable memory (e.g., EPROMs), read-only memory, and/or high-capacity storage devices (e.g., hard drives, solid state drives, etc.). In some examples, the memory 120 includes multiple kinds of memory, particularly volatile memory and non-volatile memory.

The memory 120 may be computer readable media on which one or more sets of instructions, such as the software for operating the methods of the present disclosure can be embedded. The instructions may embody one or more of the methods or logic as described herein. As an example, the instructions can reside completely, or at least partially, within any one or more of the memory 120, the computer readable medium, and/or within the processor 112 during execution of the instructions.

A request from a patient for a neurological assessment may be received at the computing device 110, such as step 202 of the process 200 shown in FIG. 2. For example, the user may request the neurological assessment by launching and interacting with a software application via the user interface 116 of the computing device 110. In some embodiments, the neurological assessment may be performed using the software application without patient interaction with a medical provider. In other embodiments, the neurological assessment may be performed using the software application in conjunction with audio and/or visual interaction with a medical provider. For example, the patient may communicate with a medical provider using conferencing software (e.g., Microsoft Teams, Zoom, Skype, BlueJeans, FaceTime, Cisco WebEx, GoToMeeting, Join.me, etc.) executing on the computing device 110, while also interacting with the software application on the computing device 110. In some embodiments, the medical provider may be able to monitor and/or control the patient's interactions with the software application, e.g., in real time.

The neurological assessment may be performed at step 204 of the process 200, which can result in the collection of assessment and sensor data. The neurological assessment may utilize the sensors 118 and/or answers from the patient to questions posed on the software application (e.g., through interaction with the user interface 116) to collect the assessment and sensor data.

In particular, various sensors 118 may directly and/or indirectly gather data regarding the patient that can be utilized in the neurological assessment. For example, a camera of the sensors 118 may track the eyes and gaze of the patient as the patient follows a target displayed on the display 114. As another example, the camera of the sensors 118 may take a picture or video of the patient and/or an injured area of the patient. As a further example, a microphone of the sensors 118 may record the speech of the patient. As additional examples, the sensors 118 may capture and/or assess facial expressions; balance sway while standing and/or walking; motion of the mouth, jaw, and/or neck; lifting of the palate in the mouth; tongue movements; tracking of finger movements; the position and/or movement of eyelids and/or eyebrows; and/or tracking of the eyeball through the eyelid while the eyes are closed. In embodiments, the data from the sensors 118 may be dynamically integrated to ensure that the process 200 can be executed on various computing devices 110 that may have differing capabilities, hardware, software, etc.

At step 204, the software application executing on the computing device 110 may also pose a variety of questions to the patient in order to gather data that can be utilized in the neurological assessment. For example, the patient may input a description of the events that occurred leading to their problem (e.g., running into a door); their symptoms (e.g., type, location, duration, severity, etc.), and/or their medical history (e.g., past illnesses and neurological events, medications being taken, etc.). In embodiments, the questions posed at step 204 may be based on standard questionnaires that ask the patient about what brings on their symptoms, sleeping behaviors, headache triggers, mental functions, and the like. Examples of such standard questionnaires may include Acute Concussion Evaluation (ACE), Sport Concussion Assessment Tool (SCAT-5, SCAT-6), Post-Concussion Symptom Scale (PCSS), Rivermead Concussion Questionnaire, Activities-Specific Balance Confidence Scale (ABC), Dizziness Handicap Inventory (DHI), Montreal Cognitive Assessment, Concussion Recovery Questionnaire (CORE-Q), Standardized Assessment of Concussion (SAC), Military Acute Concussion Evaluation (MACE), King-Devick test, and/or Neuro QOL.

As further examples, the questions posed at step 204 may be related to whether the patient: lost consciousness (and for how long); remembers what happened and/or how they got hurt; has brain fog; has fatigue along while reading, thinking, and/or moving; has menstrual cycle changes; has trouble speaking; is seeing double and/or feels that objects are moving; and/or has vertigo or feels drunk.

In embodiments, the medical provider may ask such questions and/or record the patient's answers to the questions, e.g., as the medical provider interacts with the patient through conferencing software. In general, patient data and information may be handled in accordance with applicable laws, e.g., the Health Insurance Portability and Accountability Act (HIPAA).

In embodiments, a generative artificial intelligence algorithm may be utilized at step 204 to engage in a discussion with the patient to gather information and data that is useable in performing the neurological assessment. For example, the artificial intelligence algorithm may adaptively ask questions to the patient based on the patient's previous answers. The artificial intelligence algorithm may be tuned so that the most pertinent questions are asked of the patient, and may further include guardrails to ensure that the discussion with the patient is appropriate and suitable so that the information and data is optimally gathered from the patient. In embodiments, a large language model (LLM) foundation (that is private or open source) may be used to drive a continuous model validation approach. Knowledge graphs may be created based on such LLM foundations and through use of a federated learning model to ensure regulatory compliance for specific disease conditions.

In other embodiments, data may be gathered from the patient at step 204 using generative and form-based data collection methodologies. These methodologies may help to get a better understanding of the patient's situation and circumstances.

In embodiments, a non-clinical observational assessment may be added to a model, e.g., the OMOP (Observational Medical Ontology Partnership) Common Data Model, to enhance the gathering of information and data from the patient, and to improve the assessment of any injuries the patient may have suffered. In some embodiments, a federated learning network can be utilized where the system is distributed such that the information and data is securely stored where it was generated and the analysis of the information and data may be performed centrally. Use of such a federated learning network may help to satisfy the data privacy and provenance aspects related to governance, risk management, and compliance (GRC).

Following step 204, a result may be determined at step 206 based on an analysis of the assessment and sensor data collected at step 204. In embodiments, the result determined at step 206 may be indicative of the severity of the patient's condition, and may include an urgent result, a positive non-urgent result, and a negative non-urgent result. Other types and severities of results are possible and contemplated. The result determined at step 206 may be based on whether a certain number of factors have been satisfied in the analysis of the assessment and sensor data. In embodiments, standardized guidelines may be utilized to determine the result at step 206, such as guidelines from the Centers for Disease Control and Prevention and/or other agencies.

In embodiments, an artificial intelligence algorithm may be utilized at step 206 to analyze the assessment and sensor data collected at step 204 and to determine the result. The artificial intelligence algorithm may be specific to, for example, health care, neurological impairments, and/or traumatic brain injury scenarios. The artificial intelligence algorithm may be tuned to particular industries and/or use cases so that the result that is determined at step 206 is more targeted. The result determined at step 206 may further include a summary of the findings of the analysis of the assessment and sensor data collected at step 204. In embodiments, a graphical representation may be generated at step 206 that integrates various pain and musculoskeletal ontologies and/or a risk score to provide a visualization of the findings of the analysis of the assessment and sensor data collected at step 204. The graphical representation may be utilized in conjunction with various assessment tools, e.g., Sport Concussion Assessment Tool, Brain Injury Screening Tool, etc. For example, the graphical representation may be similar to and/or based on the Circos plot concept, and may be a 2-D histogram that could be used to communicate information between a patient and a medical provider and/or a 3-D histogram that may include data to communicate with Internet-of-Things medical devices (such as heart rate monitors, sleep analysis devices, brain stimulators, spinal implants, watches, etc.).

In embodiments, the assessment data may be assessed and analyzed for particular languages (e.g., Spanish, Chinese (Mandarin and Cantonese), Tagalog, Vietnamese, Arabic, French, etc.) to build a retrieval augmented generation with a mixture of experts approach (RAG-MoE). This can assist in understanding the context of neurological diseases and conditions across different populations, e.g., for diversity, equity, and inclusion (DEI) purposes in clinical and non-clinical studies.

For example, gaze tracking test data collected at step 204 may indicate that the patient is not satisfactorily tracking a target shown during the gaze tracking test. As another example, the patient may indicate at step 204 that they are suffering from blurred vision or double vision. Further examples of factors that can be utilized to determine the result at step 206 may include whether one pupil is larger than the other; drowsiness or inability to wake up; a headache that gets worse or does not go away; slurred speech, weakness, numbness, or decreased coordination; repeated vomiting or nausea, convulsions, or seizures (e.g., shaking or twitching); unusual behavior, increased confusion, restlessness, or agitation; and/or loss of consciousness. In embodiments, existing medical data associated with the patient may be utilized in the analysis performed at step 206. Such existing medical data can be retrieved from a database associated with a medical provider or insurance company, for example, and may include past testing results, etc.

An urgent result may include when there are the number of factors satisfied in the analysis of the assessment and sensor data at step 206 exceeds a predetermined threshold, e.g., there are a relatively high number of positive factors. An urgent result may be determined if the patient should seek immediate attention and/or if their symptoms are deemed severe. For example, an urgent result may be determined at step 206 if the patient has lost consciousness and has experienced seizures. As other examples, an urgent result may be determined at step 206 if the patient has a certain number of positive factors, such as worsening headaches, drowsiness and cannot be awakened, inability to recognize people or places, unusual behavior changes, seizures, repeated vomiting, increasing confusion or irritability, neck pain, slurred speech, weakness or numbness in the arms and/or legs, and/or loss of consciousness. At step 208, it can be determined if an urgent result has been determined at step 206.

If there is an urgent result at step 208, then the process 200 may continue to step 210. At step 210, an urgent message may be displayed to the patient on the display 114 of the computing device 110. The urgent message displayed at step 210 may include instructions for the patient to seek immediate care at an emergency room or urgent care center, for example. As another example, the urgent message displayed at step 210 may include contact information and directions to the nearest emergency room or urgent care center. Further examples of the urgent message displayed at step 210 may include: advising the patient to get assistance, such as calling for emergency services; advising the patient not to move or turn their head; and/or various medical precautions, such as putting pressure or a covering on a skin abrasion or bleeding site. Following step 210, the process 200 may continue to step 220 to transmit patient data as described in further detail below.

Returning to step 208, if an urgent result has not been determined at step 208, then the process 200 may continue to step 212. At step 212, it can be determined if a positive non-urgent result has been determined at step 206. If there is not a positive non-urgent result at step 212 (i.e., there is a negative non-urgent result), then the process 200 may continue to step 214.

A negative non-urgent result may include when there are no factors satisfied in the analysis of the assessment and sensor data at step 206. A negative non-urgent result may be determined if the patient does not need medical care immediately or in the near future, e.g., there appears to be no current neurological impairment. At step 214, a message may be displayed to the patient on the display 114 to repeat the neurological assessment at a later time, e.g., in three days. The message at step 214 may also include educational information regarding neurological conditions, for example. Examples of the message displayed at step 214 may include advising the patient to schedule an appointment with a medical provider as a preventive measure. Following step 214, the process 200 may continue to step 220 to transmit patient data as described in further detail below.

Returning to step 212, if there is a positive non-urgent result, then the process 200 may continue to step 216. A positive non-urgent result may include when there are a threshold number of factors satisfied in the analysis of the assessment and sensor data at step 206, e.g., at least one factor. A positive non-urgent result may be determined if the patient appears to need medical care in the near future but not immediately. For example, a positive non-urgent result may be determined at step 206 if the patient has mild nausea and is experiencing sluggishness. As other examples, a positive non-urgent result may be determined at step 206 based on whether the patient: has sleep deficits at night; has headaches that are not worsening or progressing; has dizziness that occurs with movement; gets fatigued while reading; forgets what they are reading; and/or loses their balance when they turn.

At step 216, a potential neurological impairment may be identified as well as determining relevant medical providers that can treat the potential neurological impairment. The identified potential neurological impairment may be based on the analysis of the assessment and sensor data collected at step 204 and/or the result determined at step 206, for example. The potential neurological impairment that may be identified at step 216 may include, for example, a concussion, a stroke, or other neurological condition.

The potential neurological impairment and the relevant medical provider information may be displayed to the patient at step 218 on the display 114. The message displayed at step 218 may also include educational information and recommendations related to the potential neurological impairment, such as typical recovery times, instructions not to drive a vehicle, etc. Further examples of the message displayed at step 218 may include advising the patient regarding rest and sleep, limiting physical and mental activity, diet and fluid intake, and/or behavioral changes.

The relevant medical provider information may include contact information, links to make an appointment with the medical providers, etc. In embodiments, the relevant medical provider information may be restricted to those within a particular proximity to the location of the patient, and/or to those that accept the patient's insurance plan, for example. In embodiments, the message displayed at step 218 may include directing the patient to periodically repeat the neurological assessment in order to gather additional patient data before the patient is able to consult with a medical provider.

Following step 218 (and following step 210 and step 214 as noted above), the process 200 may continue to step 220. At step 220, patient data may be transmitted to the server 150 for storage in the database 152. The patient data may include, for example, the assessment and sensor data collected at step 204, the result determined at step 206, the factors utilized in the analysis of the assessment and sensor data, the potential neurological impairment determined at step 216, and/or the relevant medical provider information determined at step 216. In embodiments, the patient data may be encrypted and/or anonymized prior to transmission to the server 150.

The server 150 and/or the database 152 may be associated with a medical provider or insurance company, for example. The database 152 may be a relational database, although other types of database architectures may be utilized. In some embodiments, the patient data received and stored at step 220 may be utilized to further assist a medical provider to make appropriate care decisions for the patient. In other embodiments, the patient data received and stored at step 220, e.g., anonymized data, may be analyzed to determine, for example, whether particular symptoms and complaints correspond to certain neurological impairments. In further embodiments, the patient data received and stored at step 220 may be analyzed to determine relevancy with other diseases, and/or correlating the patient data with recovery timing and whether certain types of therapeutic or rehabilitation interventions may be more optimal in speeding recovery. Such analysis may include using machine learning or deep learning to optimize recovery models. The analysis may further determine the most common neurological impairments that occur after particular types of injuries and/or the neurometric findings that persist the longest after an injury.

In other embodiments, a composite risk score may be generated by the system 100 based on, for example, patient data stored in the database 152 and/or based on the assessment and sensor data collected at step 204 described above. The composite risk score may factor in environmental risks and/or neurological impairment (e.g., traumatic brain injury) risks. The composite risk score may be an assessment of the patient's risk of a neurological impairment, even before an injury may have occurred. For example, the composite risk score may be integrated with a polygenic score (that utilizes genomics, proteomics, and metabolomics) to provide a more complete picture of a patient's risk of developing pain, neurological, and/or musculoskeletal disorders. In embodiments, an application programming interface (API) may be utilized to/from the processor 112 to the server 150 or other entities to securely transfer data, scores, etc.

In an embodiment, the software application executing on the system 100 may include functionality related to assisting the decision-making for persons that are not patients or medical providers. For example, the patient data described above may be utilized to assist an attorney in determining the potential value of a lawsuit related to the potential neurological impairment of a patient, and/or whether the patient may need additional medical care or assessment of their condition to determine whether to proceed with such a lawsuit.

In an embodiment, the software application executing on the system 100 may include an analysis of the cost of assessment and treatment related to pain and neurological diseases. For example, a graphical model and/or score may be generated that assists in understanding how interventions related to pain and neurological diseases may affect the quality and/or quantity of life of patients.

Any process descriptions or blocks in figures should be understood as representing modules, segments, or portions of code which include one or more executable instructions for implementing specific logical functions or steps in the process, and alternate implementations are included within the scope of the embodiments of the invention in which functions may be executed out of order from that shown or discussed, including substantially concurrently or in reverse order, depending on the functionality involved, as would be understood by those having ordinary skill in the art.

This disclosure is intended to explain how to fashion and use various embodiments in accordance with the technology rather than to limit the true, intended, and fair scope and spirit thereof. The foregoing description is not intended to be exhaustive or to be limited to the precise forms disclosed. Modifications or variations are possible in light of the above teachings. The embodiment(s) were chosen and described to provide the best illustration of the principle of the described technology and its practical application, and to enable one of ordinary skill in the art to utilize the technology in various embodiments and with various modifications as are suited to the particular use contemplated. All such modifications and variations are within the scope of the embodiments as determined by the appended claims, as may be amended during the pendency of this application for patent, and all equivalents thereof, when interpreted in accordance with the breadth to which they are fairly, legally and equitably entitled.

1. A method for neurological analysis of a patient, comprising:
receiving a request for a neurological assessment at a processor;
collecting assessment data from the patient and collecting sensor data related to the patient, using the processor;
determining a result based on an analysis of the assessment data and the sensor data, using the processor; and
when the result comprises a positive non-urgent result:
determining a potential neurological impairment of the patient and relevant provider information, based on the analysis of the assessment data and the sensor data, using the processor;
displaying a message related to the potential neurological impairment of the patient and the relevant provider information, using the processor; and
transmitting the assessment data, the sensor data, and the potential neurological impairment from the processor to one or more of a database or a remote server.

2. The method of claim 1, wherein collecting the assessment data comprises:
displaying one or more questions to the patient on a user interface, using the processor; and
receiving one or more responses to the one or more questions at the processor from the patient through the user interface.

3. The method of claim 2, wherein displaying the one or more questions comprises:
determining the one or more questions using an artificial intelligence algorithm; and
displaying the determined one or more questions, using the processor.

4. The method of claim 3, wherein determining the one or more questions using the artificial intelligence algorithm comprises determining the one or more questions based on the received one or more responses, using the processor.

5. The method of claim 1, wherein collecting the sensor data comprises receiving the sensor data from one or more sensors that are in communication with the processor.

6. The method of claim 5, wherein the one or more sensors comprise a camera, a microphone, or an accelerometer.

7. The method of claim 1, wherein determining the result comprises determining the result based on whether one or more of the assessment data or the sensor data satisfies one or more positive factors, using the processor.

8. The method of claim 1, wherein determining the result comprises determining the result further based on existing medical data associated with the patient, using the processor.

9. The method of claim 1, wherein determining the result comprises determining the result using an artificial intelligence algorithm.

10. The method of claim 1, further comprising generating a risk score based on one or more of the assessment data, the sensor data, existing medical data, or the potential neurological impairment, using the processor.

11. A system for neurological analysis of a patient, comprising:
one or more sensors;
a user interface; and
a processor in communication with the one or more sensors and the user interface; the processor configured to:
receive a request for a neurological assessment;
collect assessment data from the patient and collecting sensor data related to the patient;
determine a result based on an analysis of the assessment data and the sensor data; and
when the result comprises a positive non-urgent result:
determine a potential neurological impairment of the patient and relevant provider information, based on the analysis of the assessment data and the sensor data;
display a message related to the potential neurological impairment of the patient and the relevant provider information; and
transmit the assessment data, the sensor data, and the potential neurological impairment to one or more of a database or a remote server.

12. The system of claim 11, wherein the processor is configured to collect the assessment data by:
displaying one or more questions to the patient on the user interface; and
receiving one or more responses to the one or more questions from the patient through the user interface.

13. The system of claim 12, wherein the processor is configured to display the one or more questions by:
determining the one or more questions using an artificial intelligence algorithm; and
displaying the determined one or more questions.

14. The system of claim 13, wherein the processor is configured to determine the one or more questions using the artificial intelligence algorithm by determining the one or more questions based on the received one or more responses.

15. The system of claim 11, wherein the processor is configured to collect the sensor data by receiving the sensor data from the one or more sensors.

16. The system of claim 15, wherein the one or more sensors comprise a camera, a microphone, or an accelerometer.

17. The system of claim 11, wherein the processor is configured to determine the result by determining the result based on whether one or more of the assessment data or the sensor data satisfies one or more positive factors.

18. The system of claim 11, wherein the processor is configured to determine the result by determining the result further based on existing medical data associated with the patient.

19. The system of claim 11, wherein the processor is configured to determine the result by determining the result using an artificial intelligence algorithm.

20. The system of claim 11, wherein the processor is further configured to generate a risk score based on one or more of the assessment data, the sensor data, existing medical data, or the potential neurological impairment.
